(19) United States
(12) Patent Application Publication
(10) Pub. No.: US 2015/0314019 A1
(43) Pub. Date: Nov. 5, 2015

(54) FUNCTIONALIZED ULTRABRIGHT FLUORESCENT SILICA PARTICLES

(71) Applicants: Igor Sokolov, Medford, MA (US); Shajesh Palantavida, Woburn, MA (US)

(72) Inventors: Igor Sokolov, Medford, MA (US); Shajesh Palantavida, Woburn, MA (US)

(73) Assignee: CLARKSON UNIVERSITY, Potsdam, NY (US)

(21) Appl. No.: 14/699,369

(22) Filed: Apr. 29, 2015

Related U.S. Application Data
(60) Provisional application No. 61/989,815, filed on May 7, 2014.
(60) Provisional application No. 61/986,201, filed on Apr. 30, 2014.

(51) Int. Cl. A61K 49/00; G01N 33/58; C09K 11/06

(52) U.S. Cl. CPC A61K 49/0052; C09K 11/06; A61K 49/0093; A61K 49/0041; G01N 33/582; C09K 2211/1018; C09K 2211/10

(57) ABSTRACT

A method for synthesizing ultrabright fluorescent silica particles with hydrophilic functional groups, comprising the steps of: (i) forming a first mixture comprising a plurality of nano-sized silica particles and a gelation agent; (ii) forming a second mixture by combining the first mixture with a surfactant, a plurality of fluorescent dye molecules, and water, wherein fluorescent dye molecules are encapsulated within a plurality of pores of the nano-sized silica particles; (iii) forming a third mixture by adding a co-source of silica to the second mixture, wherein the co-source of silica prevents leakage of the encapsulated fluorescent dye molecules from the pores of the nano-sized silica particles and provides hydrophilic functional groups to the silica particles while preserving the fluorescence of the silica particles; (iv) optional further functionalization of the obtained nanoparticles with functional molecules, exemplified by carboxylic groups and folic acid, and (v) removing excess fluorescent dye from the third mixture.

### CROSS-REFERENCE TO RELATED APPLICATIONS

This application claims priority to U.S. Provisional Patent Application Ser. No. 61/986,201, filed on Apr. 30, 2014 and entitled “Functionalized Ultrabright Fluorescent Silica Particles,” and U.S. Provisional Patent Application Ser. No. 61/989,815, filed on May 7, 2014 and entitled “Functionalized ultrabright fluorescent silica particles, methods for making and using the same,” the entire disclosures of which are incorporated herein by reference.

### FIELD OF THE INVENTION

The present invention is directed to methods for ultrabright fluorescent silica nanoparticles, and, in particular, to the preparation and use of functionalized ultrabright fluorescent silica particles suitable for suitable for many different applications.

### BACKGROUND

Fluorescence allows for the detection of very low amounts of fluorescent molecules due to a very high signal-to-noise ratio, where the background is typically non-fluorescent. Fluorescent colloids from nano- to micron-sized particles are used in a broad range of applications involving tagging, tracing, labeling, and particularly in biological applications.

Fluorescent nanoparticles are becoming increasingly popular in biomedical imaging. When functionalized with sensing molecules, these nanoparticles can be used for the detection and multiplexed imaging of specific molecules, cells, and tissues. Several groups are currently trying to develop such particles. For example, there are commercially-available fluorescent nanoparticles called quantum dots (QDs) which, although excellent in many aspects have a number of problems including long-term stability in aqueous buffers, potential toxicity, and a significant fraction of non-fluorescent QDs, among others.

In previous ultrabright fluorescence of nanoporous silica, high fluorescence came from a large number of encapsulated fluorescent dye molecules, which were dispersed inside of nanosize channels of a silica matrix. Such silica particles were created in a templated sol-gel self-assembly synthesis. It was demonstrated that physical confinement had two advantages: (a) it allowed for preservation of the quantum yield of the dye encapsulated even at very high concentrations; and (b) it made the synthesis compatible with a broad range of dyes, or combination of dyes that can withstand the synthesis. This was initially demonstrated for micron-size particles. As an example of brightness, the ultrabright silica particles can be up to two others of magnitude brighter than polymeric particles of the same size assembled with bright CdSe/ZnS quantum dots. Stable ultrabright fluorescent silica particles have been recently described in, for example, U.S. patent application Ser. No. 13/044,746, which describes the synthesis of ultrabright fluorescent silica particles (“UFSPs”).

However, the behavior of fluorescent dyes in silica material with well-defined cylindrical porosity is insufficiently studied as of yet. Traditional ways of functionalization do not work with UFSPs. Straightforward silanization chemistry substantially decreases the amount of encapsulated dye inside of the particles, and consequently decreases the fluorescent brightness. Additionally, organic solvents penetrate into nanopores and remove the dye from the particles during conjugation. Thus, standard methods of functionalization will reduce the fluorescence of the particles.

Accordingly, there is a need in the art for novel methods for the preparation of functionalized ultrabright fluorescent silica particles suitable for suitable for tagging, tracing, and labeling applications, among others.

### SUMMARY OF THE INVENTION

In accordance with the foregoing objects and advantages, methods and systems are provided for functionalization of UFSPs that preserves their fluorescent brightness. According to a first aspect, therefore, is a method for synthesizing ultrabright fluorescent silica particles with hydrophilic functional groups, the method comprising the steps of: (i) forming a first mixture comprising a silica precursor and a gelation agent; (ii) forming a second mixture by combining the first mixture with a surfactant, a plurality of fluorescent dye molecules, and water, wherein fluorescent dye molecules are encapsulated within a plurality of pores of the silica; (iii) forming a third mixture by adding a co-source of silica to the second mixture, wherein the co-source of silica prevents leakage of the encapsulated fluorescent dye molecules from the pores of the nano-sized silica particles and provides hydrophilic functional groups to the silica particles while preserving the fluorescence of the silica particles; and (iv) removing excess fluorescent dye from the third mixture.

According to an embodiment, the silica precursor is tetraethylorthosilicate (TEOS) or sodium silicate.

According to an embodiment, the gelation agent is triethanolamine (TEA).

According to an embodiment, the surfactant is cetyltrimethylammonium chloride (CTAC) or cetyltrimethylammonium bromide (CTAB).

According to an embodiment, the fluorescent dye molecules are rhodamine 6G.

According to an embodiment, the molar ratio of silica particles to gelatoin agent to surfactant to dye to water is 1:12.9:0.25:0.025:174.

According to an embodiment, the co-source of silica is aminopropyltrimethoxysilane (ATES).

According to an embodiment, the co-source of silica is aminopropyltrimethoxysilane (ATES) conjugated to folic acid.

According to an embodiment, a water-soluble carbodiimide coupling protocol is used to attach folic acid molecules to the ultrabright fluorescent nanoparticles.

According to an embodiment, the method further includes the step of conjugating the ultrabright fluorescent silica particles to folic acid.

According to an embodiment, the method further includes the step of conjugating the ultrabright fluorescent silica particles to a plurality of carboxyl groups.

According to an embodiment, the step of conjugating is performed via water-soluble carbodiimide coupling of amine-reactive succinimide esters.

According to an embodiment, the step of removing excess fluorescent dye from the third mixture comprises dialysis.

According to a second aspect is a method for labeling mammalian cells with functionalized ultrabright fluorescent silica particles, the method comprising the steps of: (i) providing functionalized ultrabright fluorescent silica particles manufactured according to the method of claim 1; and (ii) incubating the mammalian cells with the functionalized ultrabright fluorescent silica particles.

According to an embodiment, the ultrabright fluorescent silica particles are functionalized with folic acid.

According to an embodiment, the folic acid-functionalized ultrabright fluorescent silica particles preferentially label cancerous cells.

According to an aspect, a plurality of ultrabright fluorescent silica particles with hydrophilic functional groups are manufactured according to following method: (i) forming a first mixture comprising a silica precursor and a gelation agent; (ii) forming a second mixture by combining the first mixture with a surfactant, a plurality of fluorescent dye molecules, and water, wherein fluorescent dye molecules are encapsulated within a plurality of pores of the silica; (iii) forming a third mixture by adding a co-source of silica to the second mixture, wherein the co-source of silica prevents leakage of the encapsulated fluorescent dye molecules from the pores of the nano-sized silica particles and provides hydrophilic functional groups to the silica particles while preserving the fluorescence of the silica particles and (iv) removing excess fluorescent dye from the third mixture.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a schematic of a method for the functionalization of ultrabright fluorescent silica particles in accordance with an embodiment.

FIG. 2 is a TEM image of amine modified (nFA) nanoparticles with a scale bar in this image (and all images unless noted otherwise) being 50 nm, in accordance with an embodiment.

FIG. 3 is a schematic of UFSP functionalization in accordance with an embodiment.

FIG. 4A is a TEM image of FA1 particles in accordance with an embodiment.

FIG. 4B is a TEM image of FAs particles in accordance with an embodiment.

FIG. 5 is a graph of fluorescence from folic acid attached to UFSPs (FA1 and FA2), as well as the control nFA, in accordance with an embodiment.

FIG. 6 is a series of fluorescent confocal image of epithelial cancerous (cervical) cells after 15 min incubation with functionalized UBSPs in PBS buffer, wherein the bar size is 20 μm, in accordance with an embodiment.

FIG. 7 is a graph of the average fluorescent pixel intensity per cell cancer cells, where error bars correspond to one standard deviation, in accordance with an embodiment.

FIG. 8A contains representative fluorescent images along with corresponding bright field images of normal cells after incubation with FA1 and FA2 nanoparticles, in accordance with an embodiment.

FIG. 8B contains representative fluorescent images along with corresponding bright field images of precancerous/immortal cells after incubation with FA1 and FA2 nanoparticles, in accordance with an embodiment.

FIG. 8C contains representative fluorescent images along with corresponding bright field images of cancer cells after incubation with FA1 and FA2 nanoparticles, in accordance with an embodiment.

FIG. 9A is a box plot of average pixel intensities per cell for normal, precancerous, and cancerous cell cultures incubated with FA1 particles, in accordance with an embodiment.

FIG. 9B is a box plot of average pixel intensities per cell for normal, precancerous, and cancerous cell cultures incubated with FA2 particles, in accordance with an embodiment.

FIG. 10A is a series of histograms of average pixel intensities per cell for the normal, precancerous, and cancerous cell cultures incubated with FA1 particles, in accordance with an embodiment.

FIG. 10B is a series of histograms of average pixel intensities per cell for the normal, precancerous, and cancerous cell cultures incubated with FA2 particles, in accordance with an embodiment.

FIG. 11 is a series of ROC curves calculated for cancer-normal and precancerous-normal groups of cells when using either FA1 or FA2 particles for the cell labeling, in accordance with an embodiment.

FIG. 12 is a flowchart of a method for the synthesis of functionalized ultrabright fluorescent nanoparticles, in accordance with an embodiment.

### DETAILED DESCRIPTION OF EMBODIMENTS

The present disclosure describes various embodiments of the design and synthesis of nanoporous (also called mesoporous) silica particles of the diameters ranging from single nanometers to tens microns. Dye molecules are physically encapsulated inside nanochannels of mesoporous silica matrix. Due to this specific nanoenvironment, the dye molecules do not quench its fluorescence up to the concentrations which are substantially higher than the quenching concentration of free dye. Quantum yield of the dye remains sufficiently high to ensure ultrabrightness. The particle size and the dye loading were controlled by the timing of the synthesis and the amount of co-precursor of silica.

Accordingly, disclosed herein is the principle of functionalization of UFSPs while preserving brightness of the particles. The disclosed method is demonstrated using examples of functionalization of UFSPs with intermediate functional amine and carboxyl groups. This is an important step to functionalize UFSPs later with a majority of sensing molecules using standard chemistries. Here the method is shown using sensing molecules of folic acid. Folic acid receptors, for example, are overexpressed on epithelial cancer cells. The described examples are used here to exemplify the general principle of functionalization of UFSPs by introducing modified silica precursors either in the beginning or during the synthesis.

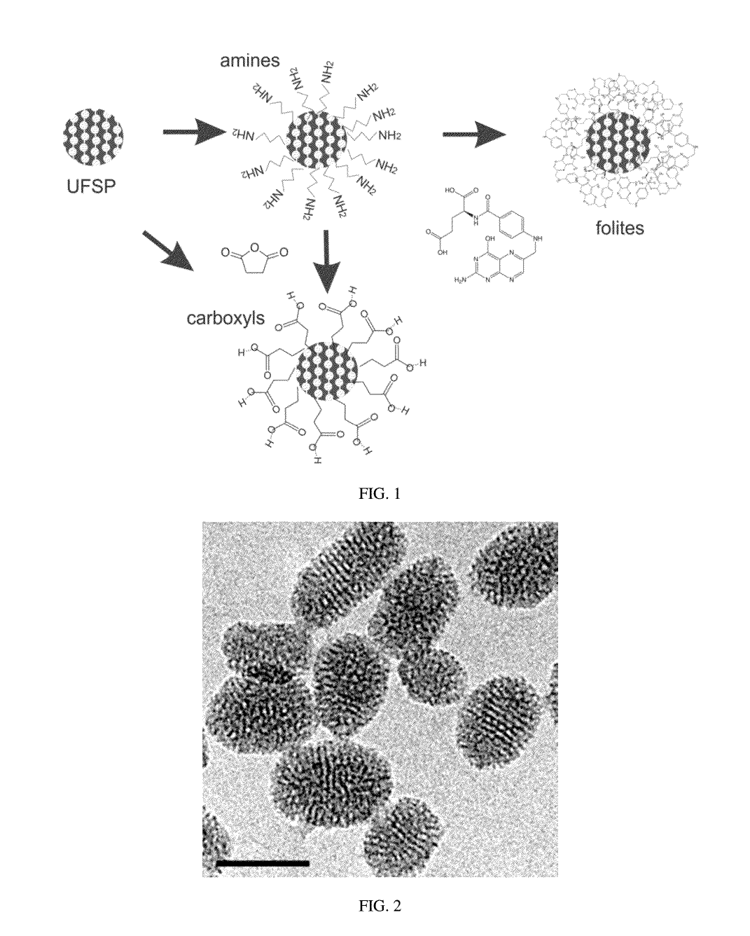

Referring to FIG. 1 is a schematic representation of the disclosed approach according to an embodiment. In this embodiment, the intermediate amine and carboxyl functional groups are introduced through the addition of organosilanes as co-precursors, functional organosilane molecules containing either amine or carboxyl groups as co-sources of silica. Having such groups on the surface will be sufficient to attach the majority of sensing molecules. At the same time, the disclosed method teaches that the addition of said functional organosilane molecules does not destroy ultrabright fluorescence of the particles.

According to another embodiment, the method is utilized to further convert amino groups to carboxyl groups by using water soluble carbodiimide coupling of amine-reactive succinimide esters. Once an amine or carboxyl group is added to the particle's surface, there are a plurality of other molecules that can be attached using standard chemistry methods. The disclosed method teaches that the addition of said amine or carboxyl groups on the particle's surface does not destroy the ultrabright fluorescence of the particles.

### EXAMPLES

Below are provided several examples which do not limit or restrict the invention in any way. Example 1 demonstrates an instance of functionalization with amine groups on UFSPs. Further functionalization with other functional molecules can be done in either aqueous or non-aqueous solution. Further functionalization in aqueous solution is trivial and involves known amine conjugation chemistry. Further functionalization in non-aqueous solution is non-trivial because organic solvents can easily wash out the encapsulated dye. Example 2 demonstrates that the methods disclosed are compatible with using organic solvents.

Reagents

According to an embodiment, the methods described or otherwise envisioned herein comprise a reaction with at least the following reagents: (1) a silica precursor which can come from either an organic or inorganic water-soluble silica source; (2) templating molecules, which can be for example any amphiphilic molecules in the concentrations above the critical micellar concentration); (3) a catalyst, either an acid or a base; (4) one or more additives to prevent the dye from leaking (including for example, organosilanes with hydrophobic groups or large polymers); and (5) one or more water-soluble florescent dyes.

According to an embodiment, the methods described or otherwise envisioned herein comprise one or more of the following chemicals for functionalization: ATES, carboxyethylsilanetriol, succinic anhydride, N-hydroxysuccinimide, 1-ethyl-3-(3-dimethylaminopropyl) carbodiimide, and/or functional molecules (such as, for example, folic acid).

Fluorescence Characterization

In the following examples, the size distribution of the nanoparticles was measured using Dynamic Light Scattering. The amount of amine groups on the nanoparticles was estimated using a modification of the fluorometric method reported by Aoki. The fluorescent complex formed between salicylaldehyde, beryllium(II) and primary amine is estimated in this method. Pure ATES was used to prepare the calibration curve. A similar method was used to measure the amount of carboxyl groups. The characteristic emission from folic acid molecules at 450 nm was used to confirm successful functionalization of folic acid on UFSPs. Measurements of zeta potential, the surface charge on UFSPs was further used as a test of functionalization of the particles' surface (in neutral acidity, amine groups have positive charge while the folic acid and carboxyl are negative). The measurement of fluorescent brightness will be done by a standard direct measurement of fluorescence from a known amount of nanoparticles as previously described.

UFSPs functionalized with folic acid were further tested for labeling of epithelial cervical cancer cells (which have overexpressed folic acid receptors). Non-functionalized UFSPs (weak positive surface charge), UFSPs functionalized with carboxyl groups (negative surface charge) and amines (positive surface charge) were used in this test as the control labels to exclude nonspecific (physical) labeling.

Particle size distributions and zeta-potential measurements were obtained using a dynamic light scattering (DLS), particle-size analyzer (Brookhaven, N.Y.) equipped with a standard 35 mW diode laser and an avalanche photodiode detector. 0.25 ml of stock solution was diluted to 3 ml with deionized water and ultrasonicated for 5 min prior to particle size measurements. Effective and most probable diameters presented are averages of three runs. To weight particles (to find the particle concentration), 0.1-0.7 mL of water suspension of UFSNP in an aluminum foil cap was dried in a vacuum chamber for 24 hours. Weighting was done five times on a CAHN29 (CAHN Instruments Inc.) balance (sensitivity 0.1 μg).

A fluorescence spectrophotometer (Varian, Cary Eclipse), and a UV-2401PC UV-Vis spectrophotometer (Shimadzu, Japan) were used to measure the fluorescence and absorbance, respectively. A Nikon Eclipse C1 confocal microscope placed on the base of a Nikon TE2000U inverted microscope (Melville, N.Y., USA) base was used to collect the fluorescence images of cells. An argon ion laser with a wavelength of 488 nm was used as the source for imaging. The optical gain for the channels was kept at 8.40 for all measurements. A 60× objective was used for the imaging. A pixel resolution of 512×512 was used to acquire the images.

The TEM images were obtained with a high resolution JEOL JEM2010 (JOEL, Japan) scanning transmission electron microscopy (200 kV accelerating voltage) equipped with a LaB₆ cathode and a Gatan SC1000 CCD camera. For TEM measurement, an adequate amount of extracted UFSNP samples was dropped onto a porous carbon film on a copper grid and then dried in vacuum.

### Example 1

### Functionalization with Amine Groups on UFSPs

According to this embodiment, tetraethyl orthosilicate (TEOS, Aldrich), γ-aminoporpyltriethoxysilane (ATES, Aldrich), cetyltrimethylammonium chloride (CTAC, 25% aqueous solution, Aldrich), triethanolamine (TEA, Aldrich), folic acid (Aldrich), N-hydroxysuccinimide (NHS, Aldrich), 1-ethyl-3-(3-dimethylaminopropyl)carbodiimide (EDC, Aldrich) and Rhodamine 6G dye (Exciton Inc.) were used in this study. All the chemicals were used without further purification. Ultrapure deionized water from a Milli-Q system was used for all synthesis, dialysis, and storage steps. Dialysis membranes of molecular weight cutoff 15 kDa (Spectra/Por regenerated cellulose) were used in all dialysis steps.

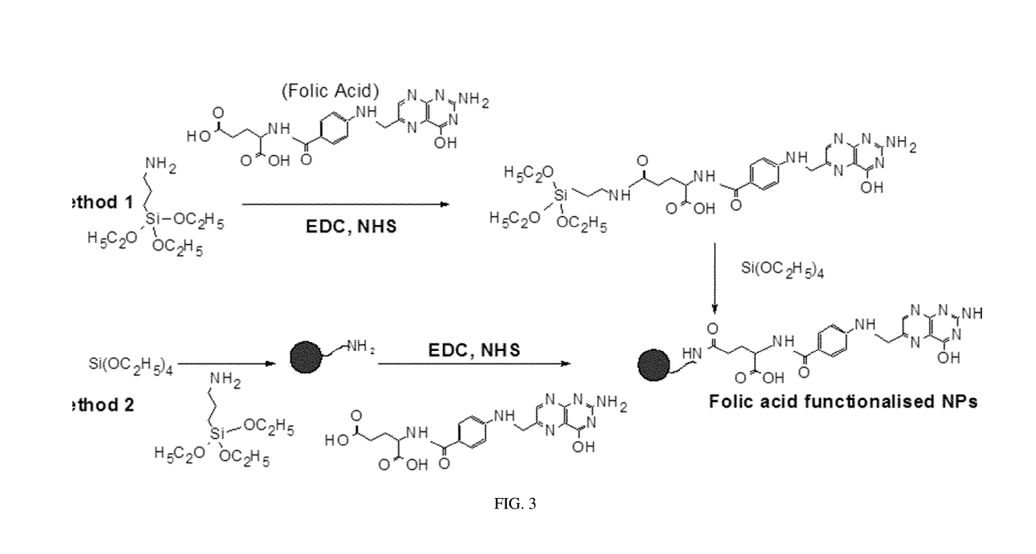

Amino-modified silica precursor was prepared in the molar ratio 1TEOS (tetraethylorthosilicate):0.025 ATES (aminopropyltrimethoxysilane). The ATES was added to the reaction mixture containing tetraethylorthosilicate, triethanolamine, cetyltrimethylammonium chloride and water in the molar ratio (1:13:0.25:174) after stirring the mixture. The obtained functionalized UFSPs had the most probable diameter of 46 nm. The particles were positively charged with a zeta potential of 10 mV. The estimated amount of amino groups was 4.5×10⁻⁸ moles/mg of silica. Assuming a density of 1.6 g/cm³ for the silica nanoparticles this would mean a high density of ˜2.2×10³ amino groups per particle. Hereafter, these UFSPs are labeled as nFA. FIG. 2 is a TEM image of the amine modified (“nFA”) particles demonstrating their porous structure.

The amount of amino groups on the nanoparticles was estimated using a modification of a known fluorometric method. The Schiff's base formed between salicylaldehyde and primary amines form a fluorescent complex with berrylium (II). The fluorescence of the formed complex is estimated in this method. Pure ATES was used for calibration. 3×10⁻³ M BeSO₄ in 10⁻² M H₂SO₄ and 10⁻² M salicylaldehyde in ethanol were used as stock solutions. 0.1 M Na₂CO₃ was used as buffer. A stock solution of 10⁻² M ATES in deionised water was prepared and used immediately for calibration. The measuring solutions were made up in a 25 ml standard flask with 10 ml buffer. The pH was adjusted, if required to 11.5 using NaOH solution. The final concentration of BeSO₄ in the measuring solution was 9.6×10⁻⁵ M and that of salicylaldehyde, 8×10⁻⁵ M. The solutions were measured after 24 h to ensure complete complexation. The fluorescence intensity at 430 nm under an excitation of 330 nm was used for preparing the calibration curve. Table 1 shows the results for the size of the particles, polidispersity and number of amino-groups.

TABLE 1
DLS results and amine concentration
in amine modified nanoparticles.

| | Most | | | Conc. of | No. of |
|---|---|---|---|---|---|
| ATES/ | Probable | Poly- | Effective | amine (nmoles/ | amine |
| TEOSmolar | Diameter | disper- | Diameter | mg of nano- | groups/ |
| ratio | (nm) | sity | (nm) | particle) | particle |
| 0.015 | 52 | 0.159 | 149 | 123 | 2350 |
| 0.025 | 46 | 0.166 | 140 | 45 | 600 |
| 0.035 | 44 | 0.190 | 140 | 56 | 650 |
| 0.05 | 49 | 0.217 | 156 | 17 | 270 |

### Example 2

### Further Functionalization with Folic Acid

This example demonstrates that carboxylic and amine modified UFSPs can be used to attach biomolecules while preserving the high brightness of the UFSPs. Folic acid is an example of such a molecule. Because folate receptors are overexpressed in the majority of epithelial cancers, this example has a direct practical application for prescreening of epithelial cancers.

A protocol for carbodiimide coupling was used to covalently attach folic acid molecules to the amine functionalized particles. Two methods of the attachment of folic acid molecules are described here. The outcome influences the brightness of the particles and extent of folic acid functionalisation. Method 1 provides particles with higher brightness. Method 2 yields particles with more folic acid functionalities. FIG. 3 shows a schematic of both methods 1 and 2.

Method 1

The folic acid functionalized UFSPs were synthesized using folic acid conjugated ATES. In a typical synthesis ATES was added to DMSO solution containing folic acid in the presence of N-hydroxysuccinimide and 1-ethyl-3-(3-dimethylaminopropyl)carbodiimide. The mix was stirred for several hours. This solution was added during UFSPs synthesis described above instead of ATES. Hereafter, these UFSPs are labeled as FA1.

As a specific example but non-restrictive example, folic acid contains two carboxyl groups, α and γ. Its conjugation with an amine group can take place through either of the carboxyl groups, but the biological activity of folic acid is retained only if it is conjugated through its γ carboxyl group. In the first approach the amine precursor ATES was conjugated to folic acid through carbodiimide coupling using EDC prior to the synthesis of nanoparticles. Briefly, the conjugation methods are follows. 28 μL of ATES was added to a 7 ml of 10% solution of folic acid in DMSO. Then, 41 mg of N-hydroxysuccinimide and 207 mg of 1-ethyl-3-(3-dimethylaminopropyl)carbodiimide were added and stirred for 3 h. This solution was added during nanoparticle synthesis instead of ATES.

Method 2

A water soluble carbodiimide coupling protocol was used to covalently attach the folic acid molecules to the amine functionalized UFSPs. A solution containing Folic acid, N-hydroxysuccinimide and 1-ethyl-3-(3-dimethylaminopropyl)carbodiimide in DMSO was stirred for several tens of minutes. Subsequently, a volume of amine modified UFSPs, prepared as described above was added to the sol and stirred further for several hours. Hereafter, these UFSPs are labeled as FA2.

As a specific but non-restrictive example, the amine functionalized nanoparticles were conjugated to folic acid directly using the same coupling scheme. As an example, a solution containing 11 mg folic acid, 6 mg N-hydroxysuccinimide and 31 mg 1-ethyl-3-(3-dimethylaminopropyl)carbodiimide in 5 ml DMSO was stirred for 20 minutes. 1 ml of the amine modified nanoparticle sol (as synthesized) was added to the previous solution and stirred for 3 h at room temperature. The resultant solution was dialyzed against deionized water for two days to remove excess reagents and unbound folic acid.

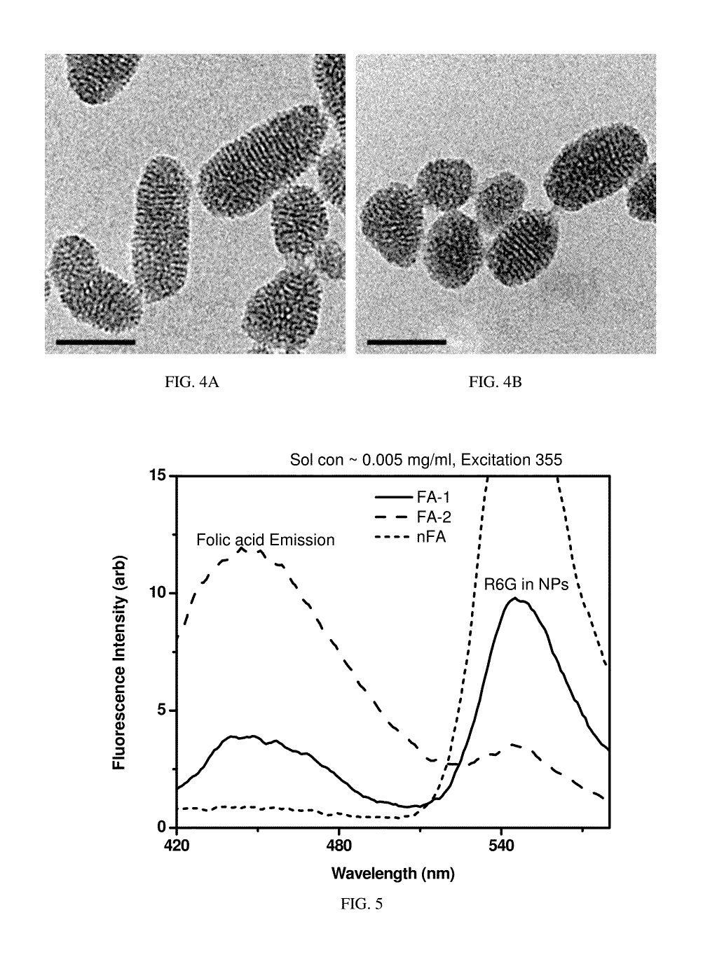

FIG. 4A is a TEM image of folic acid-modified nanoparticles produced using method 1 (FA1). FIG. 4B is a TEM image of folic acid-modified nanoparticles produced using method 2 (FA2).

The brightness of FA1 and FA2 nanoparticles and the nanoparticles of example 1 (FA) can be compared to that of a single R6G dye molecule according to the following equation:

$$Relative brightness of nanoparticles = \frac{{FL}_{NP} / {C}_{NP}}{{FL}_{R 6 G} / {C}_{R 6 G}}$$

where FL_{NP} (FL_{R6G}) is the (integral) amount of fluorescent light coming from a suspension of nanoparticles in water (solution of R6G dye) and C_{NP} (C_{R6G}) is the concentration of nanoparticles (dye concentration) in the measured suspension (solution).

As an example, the relative brightness of an nFA particle was calculated as follows. This integral intensity of fluorescence from a 3.3×10⁻⁸ M (which corresponds to the concentration of 1.99×10¹³ dye molecules/ml of R6G dye solution) was 4250 au. 3 mL of nFA stock solution of concentration 5.28 mg/mL, diluted to 10 ml had an integral fluorescence of 3969 au. Using 1.6 g/cm³ as the density of nanoporous silica and the most abundant diameter of 46 nm, this corresponds to 1.94×10¹⁰ particles per ml. The relative brightness would then be 956 dye molecules. The relative brightness of 40 nm sized alkyl modified nanoparticles reported earlier was 670 dye molecules. If we downsize the particle size of the synthesized nFA particles to 40 nm, the relative brightness would be 628 dye molecules. In comparison, FA1 particle solution containing 2.54×10¹⁰ particles per ml had integral fluorescence of 1965 au and FA2 particle solution containing 3.11×10¹⁰ particles per ml had integral fluorescence of 530 au. So the relative brightness of FA1 is 362 dye molecules and FA2 is 79 dye molecules.

This can be further compared to the brightness of water soluble quantum dots, known to be brighter than a single molecule of R6G by a factor of 20. In order to compare the brightness of the synthesized particles with water dispersible quantum dots, the nanoparticle diameter was assumed to be 30 nm for all particles. Then, the synthesized amine modified nanoparticles are 13 times brighter. The ultrabright nanoparticles functionalized with hydrophobic groups we reported earlier were 14 times brighter than quantum dots. Here we find that amine functionalisation is also successful in preventing the leakage of dyes from the pores of the nanoparticles yielding similar brightness. In a similar manner the brightness of folic acid conjugated particles were calculated. FA1 particles was found to be 8 times, and FA2 2 times brighter than quantum dots. The results are summarized in Table 2.

TABLE 2
Fluorescent parameters of the particles.

| | Brightness | Most | | | Relative |
|---|---|---|---|---|---|
| | relative to | probable | | Extinction | brightness |
| Par- | single | diameter of | | coefficient at | scaled to |
| ticle | molecule | particles | Quantum | λ = 525 nm | 40 nm |
| Type | of R6G | nm | yield % | (M cm)⁻¹ | particle size |
| nFA | 956 | 46 | 95 | 1.113 × 10⁸ | 630 |
| FA1 | 1780 | 68 | 95 | 2.07 × 10⁸ | 360 |
| FA2 | 106 | 44 | 95 | 1.23 × 10⁷ | 80 |

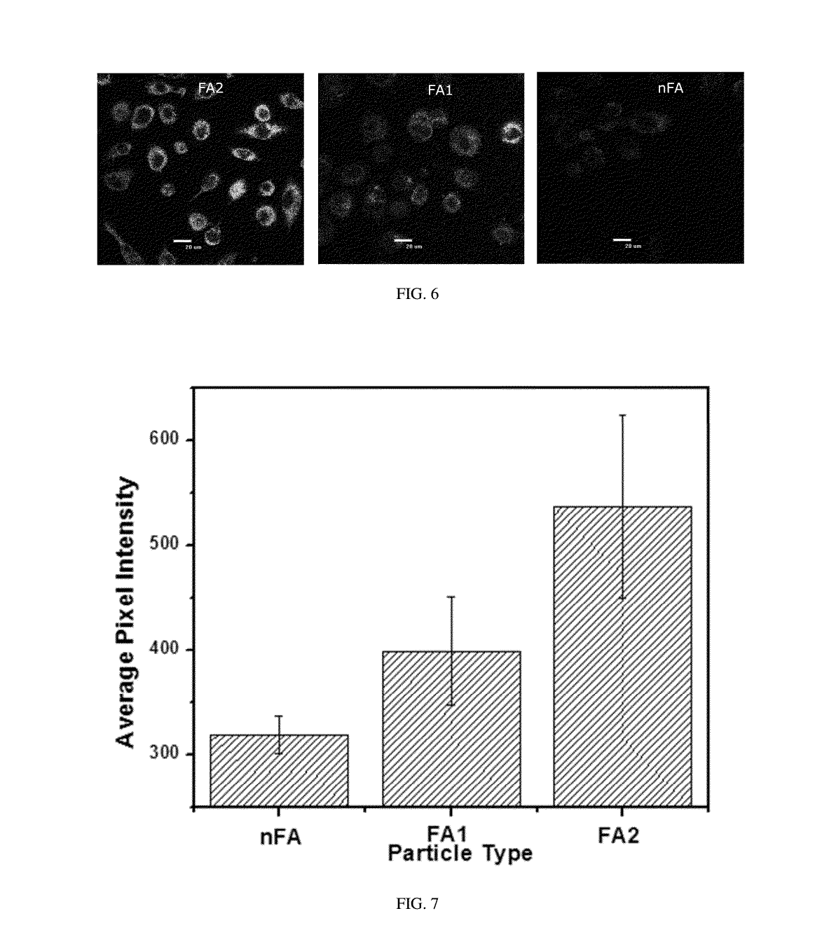

The presence of folic acid on UFSPs was detected by: (a) its characteristic fluorescence, as shown in FIG. 5; (b) the change of zeta-potential of UFSPs (becomes negative after coating with folic acid); and (c) by its preferential affinity to epithelial cancerous (cervical) cells, as shown in FIG. 6. The fluorescent brightness of UFSPs, comparing the fluorescent intensity from the samples of same concentration follow the order nFA>FA1>FA2. Comparing with the fluorescent brightness of a quantum dot, a 40 nm nFA UFSPs would be 31 times as bright, a 40 nm FA1 UFSPs would be 18 times brighter and a 40 nm FA2 UFSPs would be 4 times as bright. We did not observe the decrease of the quantum yield, only the concentration of encapsulated dye molecules decreased. Comparing the fluorescence from folic acid moieties on the nanoparticles the concentration of folic acid moieties is higher for FA2 compared to FA1.

The zeta potential value of nFA was +10 mV where as that of FA-1 was close to zero. The zeta potential measurements on FA-2 sample was −15 mV. This is also an indication of the increasing concentration of folic acid functionalization on the surface. The values were all averages of 3 measurements in pH 7 buffer.

### Example 3

### Proof of Functionalization of UFSPs with Folic Acid by Using Cancer Cells Over-Expressed with Folic Acid Receptors

Human epithelial cervical cancer cell line derived from tumors was used to test bio-activity of the functionalized particles. The details of the cell preparation were described previously. The cells were cultured in two-welled Labtek slides. The cells were washed with phosphate buffered saline (PBS) prior to incubation with nanoparticles. The incubation was done with the particular nanoparticle suspension for 15 min, and then washed twice with phosphate buffered saline (PBS), and used for imaging. The concentration of the particles was 9.3×10¹⁰ particles/mL in PBS in all cell internalization experiments. Cancerous CXT-1, 2 and 7, precancerous CX 16-1, 16-2 and 18-3 cell lines and normal HCX-132, 162 and 397 strains were used here. For the imaging study, all cells were cultured in two-welled Lab-Tek slides (Thermoscientific). The cells were used for experiments when the cells were not more than 60-80% confluent. The cells were washed with phosphate buffered saline (PBS) prior to incubation with nanoparticles. Incubation with each nanoparticle suspension was done for 15 min (1 mL of particles in PBS in concentration of 9.3×10¹³ particles/L was added to cells). To remove unbound/not internalized particles, cells were washed twice with PBS, and used for the imaging right after that.

FIG. 6 contains confocal fluorescent images of a cancerous CXT-2 cell line treated with the folate-UFSPs conjugates FA1 and FA2, and control amine-coated UFSPs (nFA). The nanoparticle stock solutions were diluted in phosphate buffered saline to obtain the same number of particles, 9.3×10¹⁰/ml in the incubating solution (calculated from the concentration of the stock and most probable diameter obtained from DLS measurements). The cells were incubated for only 15 minutes and were then washed thoroughly using phosphate buffered saline to remove any unattached nanoparticles. The imaging was performed using a 60× objective with gain of 8.40 for both channels. It is clear that the folate-labeled UFSPs are preferentially internalized by malignant cells that have overexpressed folate receptors relative to the control.

FIG. 7 is a graph of the average brightness per cell, being quantitative data shown for the cells described and exemplified in FIG. 6. This unambiguous preferential internationalization of particles by cells with folic acid receptor proves the successful functionalization of the UFSPs with folic acid.

### Example 4

### Application of Functionalized Nanoparticles for Detection of Cancer Cells

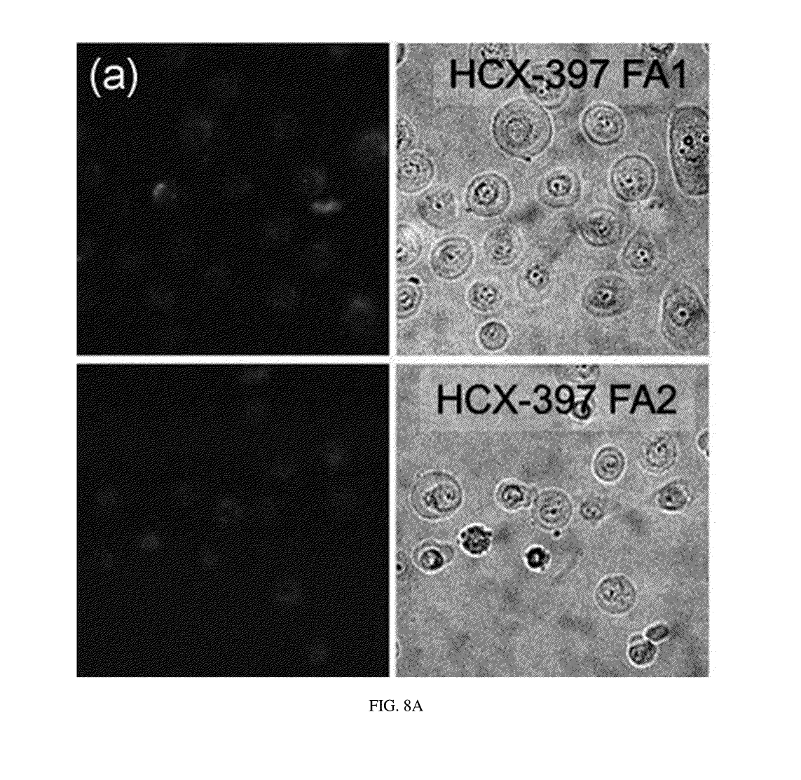

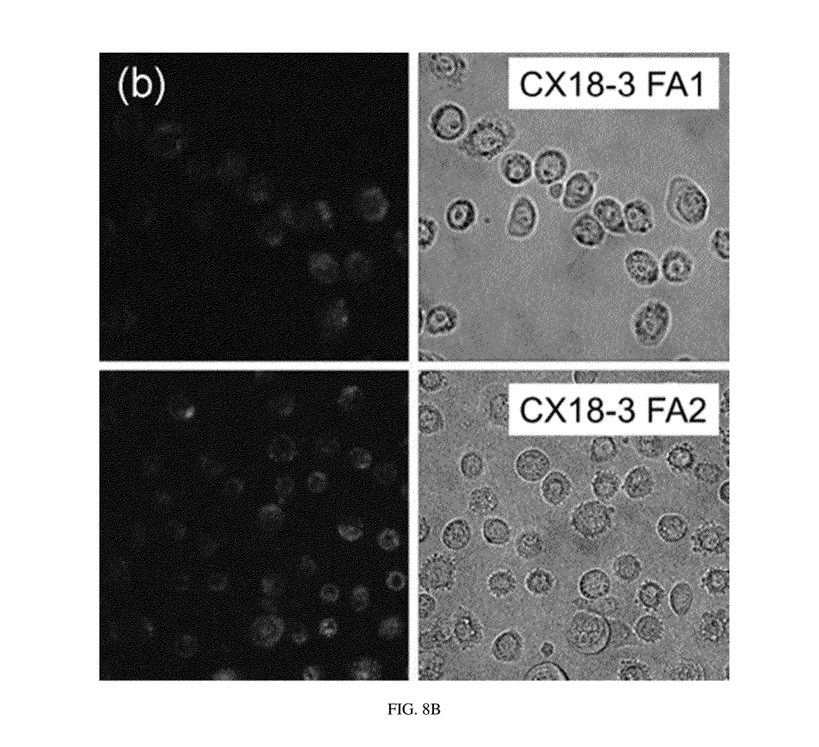

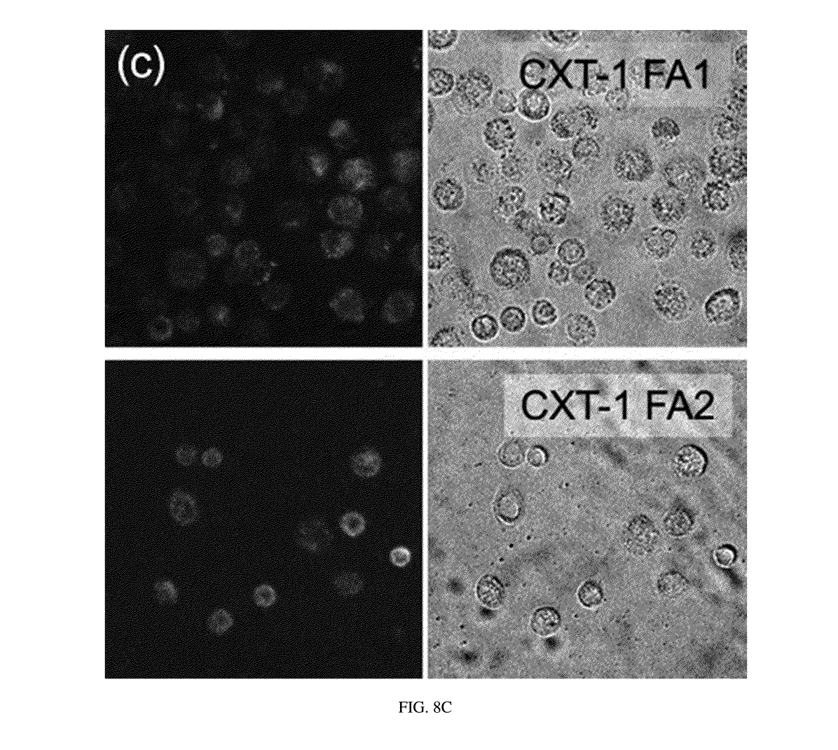

Representative fluorescent confocal images of cancer, precancerous, and normal cells after incubating with particles for 15 minutes are provided as FIGS. 8A, 8B, and 8C. It is clear that significant fluorescence is acquired by cells within this short incubation time. The high brightness of the particles must be the responsible factor. A careful visual observation shows that normal cells have also acquired some fluorescence. Nonspecific uptake of the particles by cells is well reported. It is known that the rate of nonspecific uptake of nanoparticles depends on the size of nanoparticles. The nanoparticles used in this study are in the size range where some nonspecific uptake was observed. It is also interesting that higher fluorescence intensity is observed for HCX 397 compared to HCX 162. Obviously, there is variability in the behavior of cells towards internalization of nanoparticles.

To quantify the fluorescence images of cells, the average pixel intensity of each cell was calculated from the images. About 100 cells of 20 different images were analyzed for each cell and particle type. The background pixel intensity was also measured for the areas free of cells. The background intensity per pixel was found to be in the range 35-45 arbitrary units (au) for all images irrespective of the cell and particle types. This confirms the uniformity of the imaging conditions. To exclude the background intensity from the calculation, only the pixel intensities above 50 were counted towards the cell fluorescence. Cell internalization studies have shown that the internalized particles are mostly aggregated inside vesicles and vesicles are distributed in the cytoplasm. This in turn leads to a non-uniform distribution of particles within the cell and the presence of the background pixels in the cell images. Therefore, the average pixel intensity per cell has been calculated.

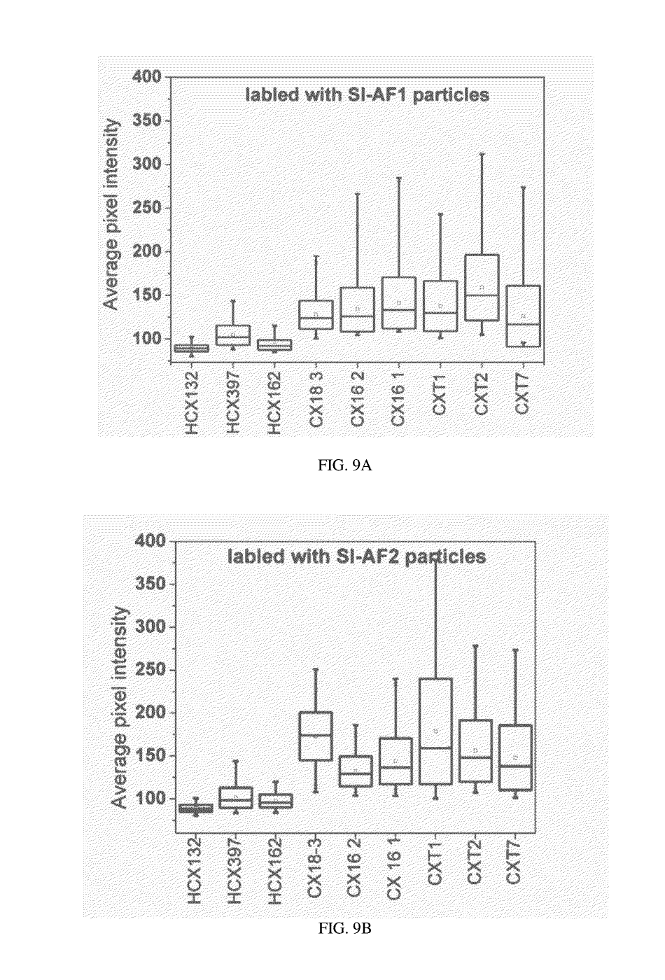

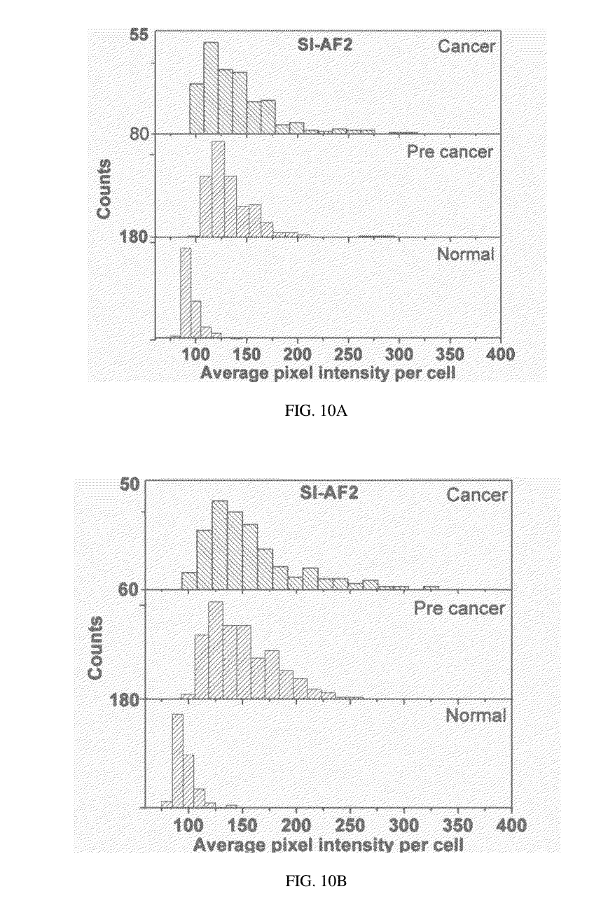

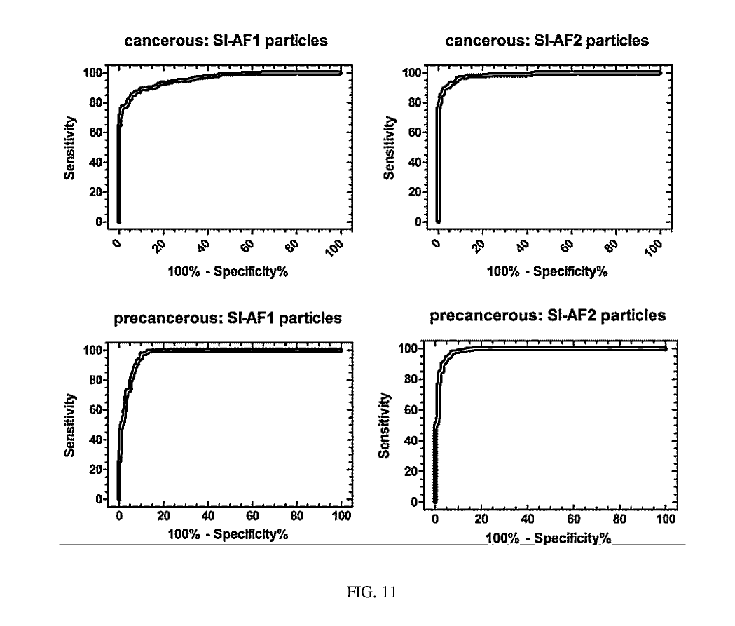

Box plots of average pixel intensity per cell obtained for the cell cultures treated with FA1 and FA2 particles are provided as FIGS. 9A and 9B. Descriptive statistics of the distributions are shown in Table 3 and FIGS. 10-11.

One can quantitatively see that the majority of cancer cells are brighter than normal cells. Precancerous cells have the intensities similar to that of cancer cells. Cancer cells have a broader distribution of average florescent intensities compared to normal cells. Segregation between intensities for cancer, precancerous and normal cells increases when using FA2 particles. It is interesting to note that out of the three normal cell strains, fluorescent intensities of one strain show a noticeable overlap with that of cancer cells. There is no overlap in the median or means of distributions any of the normal cell distributions with that of cancer or precancerous cells. The number of cells having average pixel intensity higher than 100 is much less for normal cells for both type of particles.

Comparing the means of the distributions, one can see that the cells labeled with FA2 particles have higher fluorescence intensities. Because FA2 particles are less bright than FA1 ones, this implies substantially higher internalization with FA2 particles compared to FA1 ones. Since FA2 particles have more folite molecules attached, it acts in favor to mechanism based on the folic acid receptor mediation.

Histograms of average pixel intensity of normal, cancer, and precancerous cells are provided as FIG. 11. Cancer and precancer cells have higher average pixel intensities than those for normal cells irrespective of the type of particles used. The use of FA2 particles results in a higher proportion of cells acquiring higher average intensity in the case of cancer and precancerous cells. There is some overlap of distributions for normal and the other cell types. The overlap in the distributions prevents unambiguous separation of cells.

Statistical analysis of the observed differences is done by using the Mann Whitney U tests. The nonparametric tests were chosen because of essentially non-Gaussian distributions seen in FIG. 10. The sample sizes in the Mann Whitney tests were ˜300. The U values and the significance p (one tailed) up to which the groups differed significantly are shown in Table 3. One can see that the difference between normal cells and either cancer or precancerous cells are statistically significant for p<0.0001. And the same time, the cancer and precancer distributions were not that significantly different. A similar behavior was observed in the case of FA1 particles.

TABLE 3
The results of Mann Whitney U tests comparing average pixel intensity
distributions obtained for cancer, normal and precancerous cells.
Mann Whitney U tests

| | | U | p< |
|---|---|---|---|
| FA1 | Cancer-Normal | 3360 | 0.0001 |
| | Normal-Precancerous | 2450 | 0.0001 |
| | Precancerous-Cancer | 41700 | 0.16 |
| FA2 | Cancer-Normal | 1300 | 0.0001 |
| | Normal-Precancerous | 1210 | 0.0001 |
| | Precancerous-Cancer | 40500 | 0.041 |

To understand a potential clinical value of the obtained results, receiver operating characteristic (ROC) curves were calculated. The ROC curves were calculated for cancer-normal and normal-precancerous groups. When a threshold for the average pixel intensity is specified, the classification of cells into normal and malignant is possible. If the fluorescent intensity of a cell is above the threshold, the cell is classified as malignant. The efficiency of the test to distinguish a cancer, precancerous, and normal cell can be represented in the form of the ROC curve. For an example of cancer vs normal cells, all cancer cells with intensity above the specified threshold value will be a true positive outcome, and every cancer cell below the threshold will be a false negative outcome. Similarly, every normal cell above the threshold will be a false positive outcome, and every normal cell below the threshold will be a true negative outcome. The ROC curves obtained are provided as FIG. 11. It can be seen that the ROC curves cover a high area for both types of particles, which means high efficiency of such tests. In the case of FA1, the maximum of the Youden index was obtained for the threshold of 106 when the corresponding sensitivity and specificity were 93% and 88%, respectively. In the case of FA2 particles this maximum was at the threshold of 110 when the corresponding sensitivity and specificity were 95% and 94%, respectively. The area under the curve for FA1 particle was 0.960 and that of FA2 was 0.985. (The area under a perfect test will be 1 and a worse one 0.5.)

Similar analysis on precancerous and normal cells yielded a maximum Youden index for the threshold of 108 for FA1 particles (the sensitivity and specificity were 97% and 90%, respectively). The area under the ROC curve was 0.972. In the case of FA2 particles, the maximum Youden index was obtained for the threshold of 110 (the sensitivity and specificity were 97% and 93%, respectively). The area under the ROC curve was 0.986.

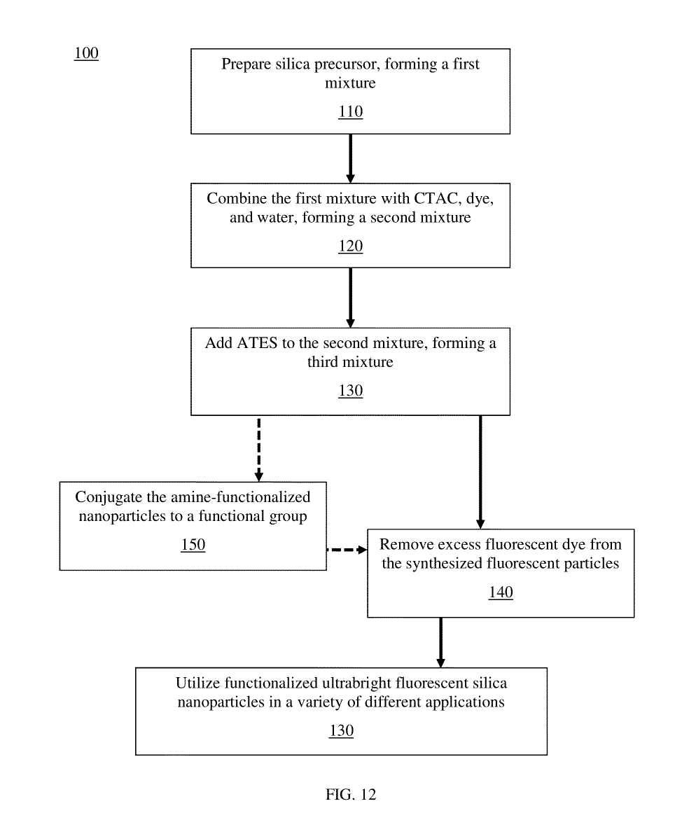

Referring to FIG. 12 is a flowchart of a method 100 for the functionalization of ultrabright fluorescent silica nanoparticles suitable for many different applications, in accordance with an embodiment.

At step 110 of the method, a silica precursor is prepared in a first mixture. For example, the silica precursor can be prepared by mixing a silica source such as tetraethylorthosilicate (TEOS) or sodium silicate, and a gelation agent such as triethanolamine (TEA). According to one embodiment, the mixture is prepared by mixing the silica source and the gelation agent in a ratio of approximately 1:12.9 and heating for approximately three hours at 90° C., with or without mixing and/or stirring.

At step 120 of the method, the first mixture, the silica precursor is combined with a surfactant—such as cetyltrimethylammonium chloride (CTAC), cetyltrimethylammonium bromide (CTAB), or an amphiphilic templating molecule, for example—a fluorescent dye, and water, to form a second mixture. According to one embodiment, a molar ratio is approximately 1 TEOS:0.25 CTAC:0.025 fluorescent dye:12.9 TEA:174 water, although variations of the molar ratio are possible. According to an embodiment, the second mixture is stirred at room temperature for 30 minutes.

The fluorescent dye may be, for example, any water-soluble fluorescent dye. As just one example, the fluorescent dye may be a member of the rhodamine family of dyes, such as rhodamine 6G (R6G). Other examples of suitable fluorescent dyes include, for example, rhodamine 640, 101, LD700, and coumarin, among many others.

At step 130 of the method, aminopropyltrimethoxysilane (ATES) is added to the second mixture to form a third mixture to add amino-group functionality to nanoparticles. The ATES can be added, for example, at a molar ratio such that the ratio between TEOS:ATES is 1:0.025. However, the molar ratio of ATES to TEOS can be varied between approximately 0.015 to 0.05, for example. The third mixture can then be stirred at room temperature for approximately 3 hours, although more or less time is possible.

According to another embodiment, the amine-functionalized nanoparticles created in step 130 are further modified with folic acid functional groups. For example, ATES is added to a DMSO solution containing folic acid in the presence of N-hydroxysuccinimide and 1-ethyl-3-(3-dimethylaminopropyl)carbodiimide. The mix is then stirred for several hours. For example, according to an embodiment, 28 μL of ATES was added to 7 ml of 10% solution of folic acid in DMSO. Then, 41 mg of N-hydroxysuccinimide and 207 mg of 1-ethyl-3-(3-dimethylaminopropyl)carbodiimide were added and stirred for 3 h. This solution is then added to the second mixture to form the third mixture as described above.

Following step 130, for example, the third mixture comprises a plurality of functionalized ultrabright fluorescent silica nanoparticles suitable for many different applications.

At step 140 of the method, excess fluorescent dye is removed from the synthesized fluorescent nanoparticles. There will often be uncaptured or unbound excess fluorescent dye present in the third mixture, and it should be removed prior to use of the fluorescent nanoparticles. According to an embodiment, the third mixture is dialyzed against deionized water until the dialyzing solution is free of fluorescent dye. This step will also remove excess reagents in the third mixture.

At optional step 150 of the method, the amine-functionalized UFSPs are conjugated with another functional group. For example, according to an embodiment, a water-soluble carbodiimide coupling protocol is used to covalently attach folic acid molecules to the amine functionalized UFSPs. A solution containing folic acid, N-hydroxysuccinimide and 1-ethyl-3-(3-dimethylaminopropyl)carbodiimide in DMSO is stirred for several tens of minutes. Subsequently, a volume of amine modified UFSPs, prepared as described above is added to the third mixture, described above, and stirred further for several hours. As an example, a solution containing 11 mg folic acid, 6 mg N-hydroxysuccinimide and 31 mg 1-ethyl-3-(3-dimethylaminopropyl)carbodiimide in 5 ml DMSO is stirred for 20 minutes. 1 ml of the amine-functionalized nanoparticles, the third mixture, is added to the previous solution and stirred for 3 h at room temperature. The method then proceeds to step 140 where excess fluorescent dye and reagents are removed.

According to yet another embodiment, the amine-functionalized nanoparticles created in step 130 or carboxy-functionalized nanoparticles created in step 150 can further be conjugated to a variety of functional groups by using standard methods.

At step 160 of the method, the fully functionalized UFSPs are utilized for a variety of different applications. For example, as described above, the functionalized UFSPs can be utilized for labeling cancer cells, which overexpress folite receptors and will recognize and internalize the folic acid-conjugated nanoparticles. Many other applications are possible.

For example, according to an embodiment, human epithelial cervical cancer cells can be incubated with the folic acid-conjugated nanoparticles, for example, for 15 min with 1 mL of particles in PBS in concentration of 9.3×10¹³ particles/L being added to the cells. To remove particles that were not, cells can be washed twice with PBS and then immediately imaged.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A method for synthesizing ultrabright fluorescent silica particles with hydrophilic functional groups, the method comprising the steps of:
forming a first mixture comprising a silica precursor and a gelation agent;
forming a second mixture by combining the first mixture with a surfactant, a plurality of fluorescent dye molecules, and water, wherein fluorescent dye molecules are encapsulated within a plurality of pores of the silica;
forming a third mixture by adding a co-source of silica to the second mixture, wherein the co-source of silica prevents leakage of the encapsulated fluorescent dye molecules from the pores of the silica particles and provides hydrophilic functional groups to the silica particles while preserving the fluorescence of the silica particles; and
removing excess fluorescent dye from the third mixture.

2. The method of claim 1, wherein the silica precursor is tetraethylorthosilicate (TEOS) or sodium silicate.

3. The method of claim 1, wherein the gelation agent is triethanolamine (TEA).

4. The method of claim 1, wherein the surfactant is cetyltrimethylammonium chloride (CTAC) or cetyltrimethylammonium bromide (CTAB).

5. The method of claim 1, wherein the fluorescent dye molecules are rhodamine 6G.

6. The method of claim 1, wherein the molar ratio of silica particles to gelatoin agent to surfactant to dye to water is 1:12.9:0.25:0.025:174.

7. The method of claim 1 wherein the co-source of silica is aminopropyltrimethoxysilane (ATES).

8. The method of claim 1, wherein the co-source of silica is aminopropyltrimethoxysilane (ATES) conjugated to folic acid.

9. The method of claim 8, wherein a water-soluble carbodiimide coupling protocol is used to attach the folic acid molecules to the ultrabright fluorescent nanoparticles.

10. The method of claim 1, further comprising the step of conjugating the ultrabright fluorescent silica particles to folic acid.

11. The method of claim 1, further comprising the step of conjugating the ultrabright fluorescent silica particles to a plurality of carboxyl groups.

12. The method of claim 11, wherein the step of conjugating was performed via water-soluble carbodiimide coupling of amine-reactive succinimide esters.

13. The method of claim 1, wherein the step of removing excess fluorescent dye from the third mixture comprises dialysis.

14. A method for labeling mammalian cells with functionalized ultrabright fluorescent silica particles, the method comprising the steps of:
providing functionalized ultrabright fluorescent silica particles manufactured according to the method of claim 1; and
incubating the mammalian cells with the functionalized ultrabright fluorescent silica particles.

15. The method of claim 14, wherein the ultrabright fluorescent silica particles are functionalized with folic acid.

16. The method of claim 15, wherein the folic acid-functionalized ultrabright fluorescent silica particles preferentially label cancerous cells.

17. A plurality of ultrabright fluorescent silica particles with hydrophilic functional groups, the silica particles manufactured according to the following method:
forming a first mixture comprising a silica precursor and a gelation agent;
forming a second mixture by combining the first mixture with a surfactant, a plurality of fluorescent dye molecules, and water, wherein fluorescent dye molecules are encapsulated within a plurality of pores of the silica;
forming a third mixture by adding a co-source of silica to the second mixture, wherein the co-source of silica prevents leakage of the encapsulated fluorescent dye molecules from the pores of the silica particles and provides hydrophilic functional groups to the silica particles while preserving the fluorescence of the silica particles; and
removing excess fluorescent dye from the third mixture.

18. The functionalized ultrabright fluorescent silica particles of claim 17, wherein the plurality of nano-sized silica particles is tetraethylorthosilicate (TEOS) or sodium silicate.

19. The functionalized ultrabright fluorescent silica particles of claim 17, wherein the gelation agent is triethanolamine (TEA).

20. The functionalized ultrabright fluorescent silica particles of claim 17, wherein the surfactant is cetyltrimethylammonium chloride (CTAC) or cetyltrimethylammonium bromide (CTAB).

21. The functionalized ultrabright fluorescent silica particles of claim 17, wherein the fluorescent dye molecules are rhodamine 6G.

22. The functionalized ultrabright fluorescent silica particles of claim 17, wherein the molar ratio of silica particles to gelation agent to surfactant to dye to water is 1:12.9:0.25:0.025:174.

23. The functionalized ultrabright fluorescent silica particles of claim 17, wherein the ATES added to the second mixture is conjugated to folic acid.

24. The functionalized ultrabright fluorescent silica particles of claim 17, wherein the functional co-source of silica added to the second mixture is aminopropyltrimethoxysilane (ATES).

25. The functionalized ultrabright fluorescent silica particles of claim 17, wherein the co-source of silica is aminopropyltrimethoxysilane (ATES) conjugated to folic acid.

26. The functionalized ultrabright fluorescent silica particles of claim 17, wherein a water-soluble carbodiimide coupling protocol is used to attach the folic acid molecules to the ultrabright fluorescent nanoparticles.

27. The functionalized ultrabright fluorescent silica particles of claim 17, further comprising the step of conjugating the ultrabright fluorescent silica particles to carboxyl groups.

28. The functionalized ultrabright fluorescent silica particles of claim 27, wherein the step of conjugation was performed via water-soluble carbodiimide coupling of amine-reactive succinimide esters.

29. The functionalized ultrabright fluorescent silica particles of claim 17, wherein the step of removing excess fluorescent dye from the third mixture comprises dialysis.
